(19) United States
(12) Patent Application Publication
(10) Pub. No.: US 2008/0215032 A1
(43) Pub. Date: Sep. 4, 2008

(54) Minimizing Metal Toxicity During Electroporation Enhanced Delivery of Polynucleotides

(75) Inventor: Dietmar P. Rabussay, San Diago, CA (US)

Correspondence Address: Biotechnology Law Group;c/o Portfolioip, P.O. Box 52050, Minneapolis, MN, 55402, US

(73) Assignee: GENETRONICS, INC., San Diego, CA (US)

(21) Appl. No.: 10/516,757

(22) PCT Filed: Mar. 25, 2003
(86) PCT No.: PCT/US03/09031

§ 371 Date: Jan. 23, 2008

Related U.S. Application Data
(60) Provisional application No. 60/367,876, filed on Mar. 25, 2002.

(51) Int. Cl. A61N 1/30; A61M 31/00; A61N 1/32; A61N 1/05; A61K 31/7088

(52) U.S. Cl. 604/501; 435/173.6; 424/185.1

(57) ABSTRACT

Methods are provided for introducing a polynucleotide into healthy tissue and generating a pulsed electric field in the tissue via invasive electrodes, resulting in enhanced delivery of the polynucleotide into cells of the tissue, while minimizing local side effects to the electroporated tissue and systemic side effects to the electroporated organism due to metal contaminants released from said electrodes. In one embodiment, the invention methods Use electrodes of gold, gold alloys, or other metal that minimize the introduction of toxic amounts of the metal into electroporated tissue. In other embodiments, the invention methods are utilized for the gene therapy by administering DNA to cells of suitable target tissue, and for the induction of an immune response by administration of a DNA vaccine.

### FIELD OF THE INVENTION

The present invention relates generally to the use of electric pulses to increase the permeability of cells, and more specifically to methods and apparatuses for the application of controlled electric fields for in vivo delivery of nucleic acids, such as genes, into cells by electroporation therapy (EPT), also known as cell poration therapy (CPI), using electrodes made of materials that do not introduce significant amounts of toxic material into the subject during said therapeutic procedure.

### BACKGROUND OF THE INVENTION

In the 1970s it was discovered that electric fields can be used to create pores in cells without causing permanent damage. This discovery made possible the insertion of small and large molecules into cell cytoplasm. As a consequence, it is known that polynucleotides, including those coding for genes and other molecules such as pharmacological compounds can be incorporated into live cells through said process which has become known as electroporation. When electroporation is applied in vitro, the genes or other molecules are mixed with the live cells in a buffer medium and short pulses of high electric fields are applied. The cell membranes become transiently porous and polynucleotides or other molecules can enter the cells.

Electroporation has been used for therapeutic processes, including the enhancement of chemotherapy of cancer. In the treatment of certain types of cancer with chemotherapeutics that act intra-cellularly, it is necessary to use sufficiently high systemic doses of drugs to achieve high enough intracellular drug concentrations to kill the cancer cells. This is frequently not possible without killing an unacceptably high number of normal cells. If the chemotherapy drug can be delivered preferentially into the cancer cells to reach high intra-cellular concentrations at low systemic concentrations, the objective of killing cancer cells without unacceptably harming normal cells can be achieved. Some of the potentially most potent anti-cancer drugs, for example, bleomycin, cannot penetrate cell membranes effectively. However, electroporation of tumors makes it possible to deliver bleomycin preferentially into the electroporated cells by making their cell membranes temporarily permeable.

Electroporation therapy treatment of cancer (tumors) typically is carried out by injecting an anticancer drug directly into the tumor and applying an electric field to the tumor between at least one pair of electrodes. The electrode configuration and the field strength must be designed in such a way that electroporation of the cells of the tumor occurs without significantly affecting surrounding normal cells. For tumors close to body surfaces, e.g., skin tumors, this can be carried out by applying non-invasive plate electrodes to opposite sides of the tumor so that the electric field between the electrodes encompasses the tumor while keeping exposure of normal tissue to the electrical field to a minimum. The electrical field between plate electrodes is rather uniform; the distance between the electrodes can be measured and a suitable voltage yielding the desired field strength according to the formula E=V/d can then be applied to the electrodes (E=electric field strength in V/cm; V=voltage in volts; and d=distance in cm). Electroporation in vivo with non-invasive electrodes is generally limited to small tumors that are close to body surfaces where the non-invasive electrodes can be placed, e.g., the skin of the organism. The treatment of large or deep-seated (internal) tumors with plate electrodes is often difficult or may sometimes be impossible, even if access to the tumor is attempted by surgical means. In addition, electrode distances exceeding approximately one cm are not practically applicable because the high voltages that have to be applied in order to achieve the desired field strengths cause unacceptable side effects. U.S. Pat. No. 5,439,440 and related patents disclose a system of electrodes for in vivo electroporation wherein the invasive electrodes may be inserted into the tumor. Such invasive electrodes allow access to desired tumors and application of desired field strengths to large tumor volumes. In related U.S. Pat. No. 5,273,525, a modified syringe for injecting molecules, including macromolecules, for electroporation utilizes needles for injection that also function as electrodes. This construction enables subsurface placement of electrodes and their use for electroporating cells situated in the tissue adjacent to and between the needle electrodes. In describing the present invention, the term “needle electrode” refers to any invasive electrode.

The use of metallic needle electrodes that are placed into healthy tissue can also be used for the purpose of accomplishing various types of gene therapy. In this application, formulated or non-formulated (“naked”) DNA is injected into normal tissue and the tissue of the injection site is then subjected to electroporation. However, optimal electroporation conditions for delivering DNA into cells differs from the optimal conditions for the delivery of relatively low molecular weight therapeutic drugs. In general, relatively longer pulses (milliseconds) at lower nominal field strengths (100-400 V/cm) are optimal for DNA delivery compared to relatively short pulses (microseconds) and higher nominal field strengths (1000-1500 V/cm) for the delivery of low molecular weight drugs (Dev, S. B. et al., IEEE Transactions on Plasma Science 28(1): 206-223 (2000)). Typical DNA delivery pulses result in higher cumulative amounts of electrical current compared to drug delivery-pulses. Higher amounts of cumulative current (Amp*sec=Coulombs, C) may result in increased electrochemical effects on the electrodes, including dissolution of certain metals of which the electrodes consist, and shedding of solid metal debris from the electrodes.

Optimal conditions for electroporation enhanced gene delivery into normal tissue for the purposes of gene therapy and DNA vaccination include pulses of 10 to 80 ms duration at nominal field strengths of 100 to 400 V/cm. However, DNA delivery can also be obtained within a broader range of conditions, e.g., 1 to 100 ms and 50 to 2000 V/cm. Comparing a commonly used DNA delivery pulse of 60 ms at 200V with a commonly used bleomycin delivery pulse of 100 μs at approximately 500V results in a 240-fold greater charge transfer (Coulombs) for the DNA delivery pulse. Thus, provided all other electrical and tissue conditions are substantially the same, the amount of electrode metal solubilized in tissue under these conditions is potentially 240 times greater when electroporation is used to deliver genes to normal tissue for purposes of gene therapy or DNA vaccination than when electroporation is used to deliver drugs to tumor tissue. The quantities of toxic metal resulting from the use of certain metal needle electrodes of various metal compositions under long-pulse conditions are often at levels toxic to tissue and the organism. In addition, metallic flakes may be shed from electrodes as a result of electrochemical processes, including corrosion, induced in the electrodes by the electroporation pulses. Dissolved metal ions and particles shedded from electrodes are thus deposited into healthy tissue where they may cause localized toxic effects. For example, when stainless steel needles were used for electroporation-enhanced delivery of genes to healthy tissue, we have observed discoloration of tissue directly at, and adjacent to, the sites of needle insertion and penetration (along needle tracks), probably due to metal contamination. We have also observed evidence of oxidation, corrosion and metal debris (flaking, scaling) on the needles themselves. The metallic contaminants may enter the lymph system and bloodstream, whereupon they can cause systemic toxicities. In treating tumors with anticancer drugs (e.g., bleomycin) and electroporation, metal contaminations are of much less concern than in gene therapy and DNA vaccination because the quantity of metal released is at least two orders of magnitude lower and toxic side effects on tumors are not considered a health risk, as opposed to side effects on healthy tissue.

The release of ferrous ions from flat stainless steel electrodes has been measured under conditions of relatively high field strength (1.2-3.0 kV/cm) and short pulses (50-500 μs) (T. Tomov and I. Tsoneva, Bioelectrochemistry 51:207-209 (2000)). The quantity of ferrous ions released was found to be proportional to the pulse duration and to the square of the field strength. No mention was made about solubilization of other components of the stainless steel electrodes, notably Chromium and Nickel, which arm of greater concern than iron in regard to cytotoxicity. Also, suggestions as to how potential toxicities originating from electrodes could be prevented were not made.

Accordingly, there is a need in the art for better methods for performing electroporation-enhanced delivery of a polynucleotide wherein needle electrodes are placed in healthy tissue. The present invention solves these and other problems in the art by providing methods for introducing a polynucleotide into cells of healthy or otherwise normal tie while minimizing toxic side effects of toxic metal released from electrodes into tissue.

### SUMMARY OF THE INVENTION

In a first embodiment of the invention, methods of electroporation are provided, said methods comprising contacting a preselected tissue with at least two needle electrodes, wherein the portion of the needle electrodes, or the surface of the needle electrodes that contacts the tissue is comprised of gold, or a gold alloy, or a metal exhibiting low toxicity when used under conditions suitable for electroporating cells for the purpose of delivering a polynucleotide into said cells. All of said metals and alloys will henceforth simply be referred to as “gold”.

In another embodiment, the methods include introducing an effective amount of at least one polynucleotide into a target tissue of a subject by a route selected from the group consisting of intramuscularly, intradermally, subcutaneously and intramucosally or via any other tissue, and generating a pulsed electric field via the at least two needle electrodes, wherein the electric field at the target tissue is of sufficient strength so as to enhance the entrance of the polynucleotide into cells of the target tissue, for example, for any gene therapy indication including DNA vaccines, as is known in the art. The pulsed electric field can be generated at substantially the same time as the introduction of the polynucleotide or after introduction of the polynucleotide as described-herein.

In another embodiment the portion of the needle electrodes that contacts the healthy tissue can comprise of gold or have at least a gold coating or plating over a shank of non-gold base metal. The term gold in the context of this document includes gold alloys that cause no unacceptable toxicity during and after application within the scope of applications described in this document. For example, the gold coating or plating can have a mean thickness of 10 μm. Optionally, at least one of the needle electrodes used in the invention methods can be hollow so that the polynucleotide is introduced via the hollow needle electrode. Although the present invention is described with respect to use of gold needles, those of skill in the art will understand that needles fashioned from, or coated with, any metal or metal containing material having material properties similar to gold, such as electrical conductivity and the lice, and which can be introduced into tissue without resulting in a toxic condition or causing discoloration of the tissue can be used for the needle electrodes in the place of the gold needles.

In another embodiment, the pulse length of the pulsed electric field is in the range from about 100 sec to about 100 msec. Preferably, the nominal field strength administered via the needles comprising gold is of sufficient strength and is delivered at substantially the same time as the introduction of the polynucleotide so as to result in the polynucleotide entering cells of the target tissue to a greater extent than in the absence of electroporation. For example, the nominal field strength can be in the range from about 50 V/cm to 5000 V/cm, preferably from about 200 V/cm to about 400 V/cm.

In still another embodiment, the invention methods are especially effective for introducing the polynucleotide into muscle or skin By use of the invention methods employing needles comprising gold, the needle electrodes do not cause substantial discoloration of the tissue by release of metal from the needle electrodes.

In yet another embodiment, the invention methods for introducing a polynucleotide into healthy tissue without introducing a toxic metal or a toxic amount of metal in the tissue are used to deliver an imnmunogenic-effective amount of at least one polynucleotide encoding an antigen into a target tissue, such as muscle or skin, to cause the polynucleotide to enter cells of the target tissue for expression therein and so as to result in generation of an immune response in the inoculated subject to the antigen encoded by the polynucleotide. Healthy tissue is contacted with at least two needle electrodes wherein the portion of the needle electrodes that contacts the tissue is gold plated or consists of gold, and a pulsed electric field is generated at the target tissue of sufficient strength so as to result in the polynucleotide entering cells of the target tissue for expression therein and so as to result in generation of an immune response in the inoculated subject to the antigen encoded by the polynucleotide.

Optionally, the immunogenicity of the polynucleotide encoding the antigen can be enhanced as compared with the immune response resulting from other modes of immunization involving administration of the polynucleotide encoding the antigen, by introducing an adjuvant-effective agent into the target tissue prior to, at the same time, or within several days of the introduction of the polynucleotide and the generation of the electric field. In the invention methods, the polynucleotide and the adjuvant-effective agent may or may not be substantially chemically associated with one another prior to the introduction thereof and, if not substantially chemically associated can be administered completely independently of one another. In a related embodiment, the use of such combinations in the invention methods provides a safe and effective approach for enhancing the immunogenicity of a wide variety of antigens without introducing a toxic amount of metal or metal ions released from needle electrodes in the healthy tissue of the subject to whom or to which the immunization protocol is administered.

Therefore, in one embodiment, the polynucleotide encoding an antigen is introduced into a target tissue of a subject by intramuscular injection. The pulsed electric field is generated at the target tissue by contacting healthy tissue with at least two needle electrodes, wherein the portion of the needle electrodes that contacts the tissue is gold. The pulsed electric field is of sufficient strength and duration and is administered at substantially the same time as the introduction of the polynucleotide so as to result in the polynucleotide entering cells of the target tissue for expression therein and so as to result in generation in the subject of an immune response to the antigen encoded by the polynucleotide; and an adjuvant-effective quantity of particles is introduced into the target tissue essentially simultaneously or within several days of the introduction of the polynucleotide and the generation of the electric field, wherein the polynucleotide and the particles are not substantially chemically associated with one another prior to the introduction thereof. The immune response resulting from the invention methods is enhanced as compared with an immune response resulting from other modes of immunization involving administration of such a polynucleotide encoding the antigen.

These and other embodiments of the present invention will readily occur to those of ordinary skill in the art in view of the disclosure herein. All publications, patents and patent applications cited herein are hereby incorporated by reference in their entirety.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods in Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); and Sambrook and Russell., Molecular Cloning: A Laboratory Manual (3rd Edition, 2000).

### DETAILED DESCRIPTION OF THE INVENTION

In describing the present invention, the following terms will be employed, and are intended to be defined as indicated below.

By “inert” is meant a stable composition that will not, on its own, react chemically with a living body in any appreciable manner when introduced into a body.

By “polynucleotide” is meant nucleic acid polymers, such as DNA, cDNA, mRNA and RNA, which can be linear, relaxed circular, supercoiled or condensed and single or double stranded. The polyaucleotide can also contain one or more moieties that are chemically modified, as compared to the naturally occurring moiety. The polynucleotide can be provided without placement into a delivery vehicle (i.e., as a “naked” polynucleotide), or in suitable vehicles, such as are known in the art. It is specifically contemplated as within the scope of the invention that the term polynucleotide for purposes of this document also encompasses oligonucleotide. In addition to the polynucleotide being administered in “naked” form, the polynucleotides may also be administered in a formulated form or modified form. For example, the polynucleotide may be formulated by mixing it with a protective, interactive, non-condensing (PINC) polymer (Fewell, J. G., el al., Gene therapy for the treatment of hemophilia B using PINC-formulated plasmid delivered to muscle with electroporation. Molecular Therapy, 3:574-583 (2000)) or the polynucleotide can be modified by attaching a peptide or other chemical entity, such as a marker molecule, to the polynucleotide (Zelphati, O., et al., PNA-dependent gene chemistry: stable coupling of peptides and oligonucleotides to plasmid DNA, Biotechniques 28:304-310; 312-314; 316 (2000)).

By “chemically associated with” is meant chemically complexed with, chemically attached to, coated with or on, adsorbed to, or otherwise chemically associated. For instance, nucleic acid that is coated on or adsorbed to particles is chemically associated with the particles. Association can mean covalent or non-covalent bonds.

By “dermal tissue” is meant epidermis and dermis below the stratum corneum.

By “intradermal” and “intradermally” is meant administration into, but not on the surface of, dermal layers of the skin. For example, an intradermal route includes, but is not limited to, tumors of dermal cells.

By “intramuscular administration” and “intramuscularly” is meant administration into the substance of the muscle, i.e., into the muscle bed.

By “intramucosal administration” and “intramucosally” is meant administration into the mucosa or mucous tissue lining various tubular structures, including but not limited to the aero-digestive and urogenital tracts.

By “subcutaneous administration” and “subcutaneously” is meant administration into tissue underlying the skin.

By “immunization” is meant the process by which an individual is rendered immune or develops an immune response.

By “antibody” is meant an immune or protective protein evoked in animals, including humans, by an antigen and characterized by a specific reaction of the immune protein with the antigen.

By “at substantially the same time” with reference to the timing of the coadministration of the polynucleotide and the pulsed electric field, is meant simultaneously, or within minutes to hours of administration of each other.

By “antigen” is meant a molecule that contains one or more epitopes that will stimulate a host's immune system to elicit a humoral antibody response or cellular antigen-specific immune response when the antigen is presented. Normally, an epitope will include between about 3-15, generally about 5-15, amino acids. For purposes of the present invention, antigens can be derived from any of several known viruses, bacteria, parasites and fungi. The term also is intended to encompass any of the various tumor antigens. Furthermore, for purposes of the present invention, an “antigen” includes those with modifications, such as deletions, additions and substitutions (generally conservative in nature), to the native sequence, so long as the protein, polypeptide or polysaccharide maintains the ability to elicit an immunological response. These modifications may be deliberate, as through site-directed mutagenesis, or may be accidental, such as through mutations of hosts that produce the antigens.

An “immune response” to an antigen or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytolines, chemokines and other such molecules produced by activated T-cells and/or other white blood cells, including those derived from CD4+ and CD8+ T-cells.

An invention method “enhances immunogenicity” of the polynucleotide encoding an antigen when it hastens the appearance of an immune response (i.e., enhances kinetics of the immune response) or possesses a greater capacity to elicit an immune response than the immune response elicited by an equivalent amount of the polynucleotide without the particle/pulsed electric field adjuvant effect. Thus, the method for inducing an immune response may display “enhanced immunogenicity” because the antigen produced is more strongly immunogenic or because a lower dose of polynucleotide encoding the antigen is sufficient to achieve an immune response in the subject to which it is administered, or because an efficient immune response, e.g., as manifested by, but not limited to antibody titer, is reached more rapidly after administration. In the present invention, the enhanced immune response preferably includes the advantage that the kinetics of the immune response is faster as evidenced by faster appearance of an immune response, e.g., as evidenced by a rise in antibody titer, than in other immunization protocols. Such enhanced immunogenicity can be determined by administering the polynucleotide composition and pulsed electric field, or the polynucleotide and the particles as controls to animals and comparing immune response against the invention methods using standard assays such as radioimmunoassay and ELISAs, as is well known in the art.

The term “adjuvant-effective quantity” as applied to the adjuvant used in the invention methods refers to sufficient quantity of the adjuvant to provide the adjuvant effect for the desired immunological response and corresponding therapeutic effect. The exact amount required will vary from subject to subject, depending on the species, age, and general condition of the subject, the severity of the condition being treated, and the particular polynucleotide encoding the antigen of interest, mode of administration, e.g., whether to muscle or skin, the type of the adjuvant, and the like. An appropriate “effective” amount in any individual case may be determined by one of ordinary skill in the art using routine experimentation.

The compositions comprising the polynucleotide encoding an antigen will comprise an “immunogenic-effective amount” of the polynucleotide of interest. That is, an amount of polynucleotide will be included in the compositions that, when the encoded antigen is produced in the subject, in combination with the particles and the pulsed electric field, will cause the subject to produce a sufficient immunological response in order to prevent, reduce or eliminate symptoms. An appropriate effective amount can be readily determined by one of skill in the art. Thus, an “immunogenic-effective amount” will fall in a relatively broad range that can be determined through routine trials.

As used herein, “inducing an immune response” refers to any of (i) the prevention of infection or reinfection, as in a traditional vaccine, (ii) the reduction or elimination of symptoms, and (iii) the substantial or complete elimination of the pathogen in question. Thus, the methods for inducing an immune response may be effected prophylactically (prior to infection) or therapeutically (following infection).

By “pharmaceutically acceptable” or “pharmacologically acceptable” is meant a material which is not biologically or otherwise undesirable, i.e., the material may be administered to an individual along with the particle adjuvant formulations without causing any undesirable biological effects or interacting in a deleterious manner with any of the components of the composition in which it is contained.

By “subject” is meant any mammal, including, without limitation, humans and other primates, including non-human primates such as chimpanzees and other ape and monkey species; farm animals such as cattle, sheep, pigs, goats and horses; domestic mammals such as dogs and cats; laboratory animals including rodents such as mice, rats and guinea pigs, domestic pets, farm animals, such as chickens, and the like. The term does not denote a particular age. Thus, both adult and newborn individuals arc included among the subjects who can be treated according to the invention methods. The invention methods described herein are intended for use in any of the above mammalian species, since the immune systems of all of these mammals operate similarly.

According to the embodiments of the present invention, when gold needles are used to generate a pulsed electric field in healthy tissue, even though the pulse length is up to 100 msec in length, as is advantageous for introduction of polynucleotides and other molecules used in gene therapy and as DNA vaccines, creation of a toxicity-causing release of metal or metal ions from the needles into the treated tissue can be avoided.

For example, to cause electroporation of cells in muscle tissue for purposes including gene therapy and DNA vaccination, the pulsed electric field used in the invention methods will have a nominal electric field strength from about 50 V/cm to about 2500 V/cm, preferably about 200 V/cm to about 400 V/cm. The length of pulses used in the pulsed electric field delivered to muscle will be in the range from about 1-100 milliseconds (msec), preferably 20-60 msec and about 1-6 pulses will be applied at a frequency of 0.1-100 Hz The waveform of the electric pulses can be monopolar or bipolar. For the invention method of delivering a polynucleotide for gene therapy of DNA vaccination into skin, the pulsed electric field will be developed with from 1 to about 12 pulses of 50V to 200 V each, lasting from about 100 microseconds to 100 msec each, at 0.1-1000 Hz.

For generation of an electric field in muscle at substantially the same time as introduction of a DNA vaccine or a polynucleotide intended for a gene therapy indication, needle electrodes comprising two, four, or six electrodes are preferred. Gold or gold coated electrodes configured into pairs, opposed pairs, parallel rows, triangles, rectangles, squares, or any other suitable geometry are contemplated.

For generation of an electric field in skin at substantially the same time as introduction of a DNA vaccine, various invasive electrodes can be used. For electroporation applied to the surface of the skin, short needle electrodes from less than one millimeter to several millimeters in length so as to penetrate the stratum corneum and epidermis and dermis to certain depths, arc preferred. By contrast, for electroporation applied to muscle, longer needle electrodes are preferred.

Several presently preferred conditions for providing electroporation in practice of the invention methods are provided in Table 1 below, wherein the needles used for electroporation comprise gold such that generation of an electric field in healthy tissue using the needle electrodes does not result in introducing a significant amount of toxic metal from the needle electrodes in the tissue.

TABLE 1

| Site of | Type of | Field | Number | | Applied | Frequency |
|---|---|---|---|---|---|---|
| delivery | electrode | strength | of pulses | Pulse length | voltage | in Hz |
| Muscle | 2-needle | Low | 1-3 | Long | N/A | 0.1-10 |
| | electrode | 150-200 V/cm | identical | 60 msec | | |
| | | | pulses | | | |
| Muscle | 4 needle | Low | 1-3 | Long | N/A | 0.1-10 |
| | electrode | 150-200 V/cm | identical | 60 msec | | |
| | | | pulses | | | |
| Muscle | 6 needle | Low | 6 | Long | N/A | 0.1-10 |
| | electrode | 100-200 V/cm | identical | 20-60 msec; | | |
| | | | pulses w/ | | | |
| | | | polarity | | | |
| | | | reversal | | | |
| Into skin | Short | Low | 1-6 | Long | | 0.1-50 |
| cells | needle | 100-250 V/cm | identical | 100 μsec-60 msec | | |
| | | | pulses | | | |

The methods of the present invention can be practiced with mucosal tissues as the target tissues, such as buccal and nasal membranes. The parameters for application of the electric charge are substantially the same as those set forth herein for skin tissue. Polynucleotides may be delivered to mucosal tissue and cells, or cells underlying the mucosa by injecting polynucleotide in naked, formulated or modified form into the mucosa or by topical application, followed by electroporation with minimally invasive needle electrodes comprising gold, such as electrodes consisting of multiple, short-needle electrodes (U.S. Pat. No. 5,810,762; Glasspool-Malone, J., et al. Efficient nonviral cutaneous transfection. Molecular Therapy 2:140-146 (2000); Zhang, L., et al. Enhanced delivery of naked DNA to the skin by non-invasive in vivo electroporation Biochem. Biophys. Acta 1572(1): 1-9 (2002)). One of skill can perform straightforward experiments to determine the optimal conditions for delivery of a DNA vaccine to a specific mucosal tissue.

The methods described herein provide a means for treating a variety of malignant cancers. For example, the invention methods can be used to mount both humoral and cell-mediated immune responses to particular proteins specific to the cancer in question, such as an activated oncogene, a fetal antigen, or an activation marker. Such tumor antigens include, without limitation, any of the various MAGEs (melanoma associated antigen E), including MAGE 1; 2, 3, 4, etc. (Boon, T. Scientific American (March 1993):82-89); any of the various tyrosinases; MART 1 (melanoma antigen recognized by T cells), mutant ras; mutant p53; p97 melanoma antigen; CBA (carcinoembryonic antigen), among others. It is readily apparent that the subject invention can be used to prevent or treat a wide variety of diseases.

The compositions will generally include one or more “pharmaceutically acceptable excipients or vehicles” such as water, saline, glycerol, polyethylene glycol, hyaluronic acid, ethanol, etc. Additionally, auxiliary substances, such as wetting or emulsifying agents, pH buffering substances, and the like, may be present in such vehicles.

The following are illustrative examples of specific embodiments for carrying out the present invention. Gold needles can be used to administer the pulsed electric field in the following examples, which are offered for illustrative purposes only, and are not intended to limit the scope of the present invention in any way.

### EXAMPLE 1

The objective of this experiment was to determine quantitatively the effect of electroporation pulses on the integrity of stainless steel needle electrodes. This experiment was prompted by observations of discoloration of tissue at the needle tracks and the fact that said electrodes showed signs of deterioration after being used for in vivo electroporation purposes under pulse conditions preferably used for polynucleotide delivery. The signs of deterioration including roughening, pitting and flaking of the originally smooth and shiny needle surface, change of color from silvery to dark brown and black, and dulling of the sharp needle tips. These signs of deterioration increased with the number of pulses which were delivered through these electrodes to the point that a decrease of the needle diameter became visible with the naked eye after the delivery of approximately 30 pulses. The generally held assumption that stainless steel electrodes are chemically inert and biocompatible under electroporation conditions was therefore questionable.

The stainless steel from which the needles were manufactured had a composition of approximately 74% iron (Fe), 18% chromium (Cr) and 8% nickel (Ni). It is known that even small quantities of Cr and Ni can cause local tissue toxicities and that systemic toxicities can result when Cr and Ni in soluble form are distributed throughout the body via the blood stream and the lymphatic system. To determine the amount of metal shed from stainless steel needle electrodes, six samples of six-needle array electrodes described earlier (G. A. Hofmann et al., Critical Reviews in Therapeutic Drug Carrier Systems 16:523-569 (1999)) were immersed into USP-grade phosphate-buffered saline to a depth of 13 mm and 200V square wave electroporation pulses of 25 ms and 60 ms duration, respectively, were applied at 2 Hz. Control samples were prepared exactly the same way except that no electrical pulses were delivered. Solid debris observed in electroporated samples were dissolved by the addition of acid. The amount of metal ions in the test samples was then determined by Inductive Coupled Plasma Mass Spectrometry (ICPMS). The results are summarized in Table 2. The surprising results of this study included that the total amount of metal detected in the assay solutions was about 5-fold greater than the normal amount expected to be solubilized from calculations based on established laws of electrochemistry and measured charge transfer (Coulombs applied). A likely explanation for this surprising finding is that the amount is due to dislocation of solid particles from the surface of the electrodes. It appears that stainless steel which is considered to be relatively inert and corrosion resistant not only is affected by known electrolytic processes but also suffers structural disintegration at its surface which results in shedding of solid metal particles. This unexpected phenomenon could be caused by the high current density or high field strength to which the needle electrodes are exposed for tens of milliseconds. It is interesting that pulses of higher field strength and current density but of shorter duration, such as used for the in vivo delivery of bleomycin, exert much lesser destructive effect on these stainless steel needles than the electrical conditions used for in vivo polynucleotide delivery. In other words, the high amount of metal released from the electrodes, which is in several-fold excess to what one skilled in the art of electrochemistry would expect, is a novel finding that has direct consequences for the use of such electrodes in electroporation therapy applications.

The valence state of the solubilized metals has not been investigated. However, it must be taken into consideration that during in vivo electroporation higher valence states of Cr and Ni may be generated whose toxicity is greater than the toxicity of lower valence states and therefore the toxic effect of the bio-available quantities of metal ions may be greater than if Ni and Cr are assumed to be at the divalent state.

Linear extrapolation of the metal shedding results suggests that after approximately three thousand 60 ms pulses, or approximately seven thousand 25 ms pulses the electrode needles would be completely disintegrated (22 gauge needle, length 13 mm). However, linear extrapolation may not be valid in this case and needle deterioration may actually accelerate with increasing pulse number.

TABLE 2

| | | | | Metal | | |
|---|---|---|---|---|---|---|
| | | Duration | Charge | solubilized milligrams | | |
| Sample | Voltage V | ms | mC | Fe | Cr | Ni |
| 1 | — | — | — | negligible | | |
| 2 | 200 | 25 | 128 | 0.104 | 0.024 | 0.012 |
| 3 | 200 | 60 | 224 | 0.222 | 0.061 | 0.025 |
| 4 | — | — | — | negligible | | |
| 5 | 200 | 25 | 128 | 0.103 | 0.024 | 0.012 |
| 6 | 200 | 60 | 224 | 0.225 | 0.062 | 0.026 |

### EXAMPLE 2

The objective of this study was to find needle electrodes of appropriate composition which would result in the shedding of relatively low amounts of toxic metal under electroporation conditions employed for the in vivo delivery of polynucleotides. As shown in Example 1, stainless steel needle electrodes can shed substantial amounts of toxic metal, both in the form of metal ions and metal particles. Of primary concern are Cr and Ni. Iron is of lesser concern. Both Cr and Ni are known to be able to cause a variety of toxicities, well described in the medical literature. Ni is also known to cause allergies in a significant number of people.

Needle electrodes for electroporation must meet a number of requirements. Their mechanical properties must be such that they can easily be inserted into muscle and other tissue, through skin, without having to apply undue pressure. The needles must be stiff enough so as to not bend while being inserted (the needles in needle arrays must remain parallel to each other) and they must not be brittle so as not to break or shatter when hard obstacles (e.g. bone) arm encountered, or when accidentally subjected to bending forces. Needles must also be easy and economical to manufacture. In addition, needles must be sufficiently electroconductive and biocompatible. Any electrolytic products or particles originating from the needles during electoporation must not give rise to significant local or systemic toxicities.

We tested a variety of needles, consisting of either tungsten or stainless steel as the base metal, coated with gold using different plating procedures. The testing procedure involved mechanical tests as well as electrochemical tests. Electrochemical tests were performed by immersing previously unused needle arrays to a depth of 1 cm into phosphate-buffered saline (USP grade) and applying six 200V square wave pulses at a frequency of 2 Hz and of various millisecond pulse durations. Polarity of the electrodes was reversed after each pulse. After the pulses, the saline solution was analyzed for gold (Au), tungsten (W), Fe, Ni and Cr by ICPMS. The analytical results of some of the needles tested are summarized in Table 3. Compared to the stainless steel electrodes, needles of ss/Gold #3 showed reduced Cr, Fe and Ni in solution after electroporation. The concentration of gold was 897 ppb, which corresponds to a negligible level of toxicity. Needles of the W/Gold #3 type (tungsten needles coated with gold) showed negligible levels of Cr, Fe and Ni. The tungsten and gold concentrations were insignificant from a toxicity point of view. From these results it can be concluded that the toxicity associated with the use of stainless steel needles in electroporation therapy can be significantly reduced by choosing materials that possess mechanical and electrical properties suitable for needle electrodes, that are easy and economical to manufacture, and that impart minimal toxicity on the subject being treated when used for electroporation therapy procedures.

TABLE 3

| | Metal in Saline | | | | |
|---|---|---|---|---|---|
| | after electroporation | | | | |
| | pulses [nanograms/milliliter, ppb] | | | | |
| Needle Type | Cr | Fe | Ni | W | Au |
| Stainless steel (ss) | 5,600 | 19,700 | 2,510 | 6 | 2 |
| ss/Gold #3 | 886 | 1,080 | 1,540 | 1 | 897 |
| W/Gold #3 | 3 | 120 | 3 | 88 | 1920 |

### EXAMPLE 3

The objective of this study was to assess the biocompatibility of electrodes of various metal composition when tested under conditions mimicking in vivo electroporation for the purpose of delivering polynucleotides into target cells. Three different types of needles were evaluated, together with a saline control. Unplated 304 stainless steel needles, gold-plated 304 stainless steel needles, and gold-plated tungsten needles were tested in the form of needle arrays. In these arrays, four needles were mounted in a nonconductive handle at the four corners of a 0.86×0.5 cm rectangle. The four needles were connected to a pulse generator in such a way that two opposing needle pairs each were pulsed at the same time. The distance between the + and − electrodes in each pair was 0.86 cm, the distance between the two pairs was 0.5 cm. The four needles of each array were immersed into 12 ml each of saline to a depth of 2.8 cm and pulsed 10 times at 200 V for 60 msec each, with a square wave pulse at 2 Hz. After pulsing, 6 ml of each sample were used for cytotoxicity testing and 6 ml for chemical analysis. For cytotoxicity testing, each 6 ml sample was mixed with 2 ml 4×MEM (minimal essential medium) with 50% calf serum, and the pH was adjusted with NaHCO₃ to 7.2 (“Test Solution”). Six-well cell culture plates were seeded with L929 cells and cells were grown in 1×MEM with 10% calf serum at 37° C. in a 5% CO₂/air atmosphere to 80-90% confluency. The medium was removed from the wells and triplicate samples of 2 ml each of each Test Solution were added per well. Plates were incubated for 48±3 hrs by microscopic evaluation after neutral red staining for viable cells. The scores for each set of triplicate samples was averaged and recorded as such. Negative and positive controls of the cytotoxicity assay were also run. Table 4 describes the criteria used in scoring the results. The cytotoxicity scores are summarized in Table 5. Based on the results obtained, the unplated stainless steel needles showed substantial cytotoxicity whereas the gold-plated stainless steel and tungsten needles both ehhibited no detectable cytotoxicity.

TABLE 4

| Grade | Reactivity | Description of Reactivity Zone |
|---|---|---|
| 0 | None | Discrete intra-cytoplasmic granules, no cell lysis |
| 1 | Slight | Not more than 20% of the cells are rounded, loosely |
| | | attached, and without intra-cytoplasmic granules; |
| | | occasional lysed cells are present. |
| 2 | Mild | Not more than 50% of the cells are rounded and |
| | | devoid of intra-cytoplasmic granules; no extensive |
| | | cell lysis and empty areas between cells. |
| 3 | Moderate | Not more than 70% of the cells are rounded and/or |
| | | lysed. |
| 4 | Severe | Nearly complete destruction of the cells. |

TABLE 5

| | Grade, | Grade, | Grade, | Average |
|---|---|---|---|---|
| Sample Identification | Trial #1 | Trial #2 | Trial #3 | Grade |
| Saline Control | 0 | 0 | 0 | 0 |
| Gold Plated Tungsten | 0 | 0 | 0 | 0 |
| Gold Plated 304 Stainless Steel | 0 | 0 | 0 | 0 |
| Unplated 304 Stainless Steel | 4 | 4 | 4 | 4 |

We claim:

1. A method of decreasing toxic metallic contaminants derived from a metallic electrode inserted in tissue, or cells suspended in a medium, undergoing treatment by electroporation, wherein said electroporation is carried out using electrodes within said tissue or within said medium comprising:
a) contacting said tissue or medium with electrodes selected from the group consisting of gold electrodes, gold plated electrodes, and gold alloy electrodes; and
b) charging said electrodes with an electric pulse capable of electroporating said tissue or cells.

2. A method according to claim 1 wherein said electrodes further comprise needles sufficient to penetrate said tissue.

3. A method according to claim 1 wherein said pulse is selected firm a square pulse, a bipolar pulse, and a rectangular pulse.

4. A method according to claim 1 wherein the nominal field strength of said pulse is from 10 to 1500 V/cm.

5. A method according to claim 1 wherein the duration of the pulse is between 1 and 100 ms.

6. A method according to claim 1 wherein the frequency with which multiple pulses are applied is between 0.1 and 1000 Hz.

7. A method of electroporating polynucleotides in tissues of a subject and decreasing toxic metallic contaminants derived from a metallic electrode inserted in said tissue, wherein said electrporation comprises:
a) contacting said tissue with electrodes selected from the group consisting of gold electrodes, gold plated electrodes, and gold alloy electrodes; and
b) charging said electrodes with an electric pulse capable of electropoating said polynucleotides into cells of said tissue, said electric pulse having a nominal field strength of between 10-1500 V/cm.
